(19) United States
(12) Patent Application Publication
(10) Pub. No.: US 2006/0115908 A1
(43) Pub. Date: Jun. 1, 2006

(54) Multiplexed analyses of contaminant-laden gas in a particle impact collector

(75) Inventor: Thomas W. O'Brien, Fairfax, VA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TETRACORE, INC., Rockville, MD (US)

(21) Appl. No.: 11/271,040

(22) Filed: Nov. 10, 2005

Related U.S. Application Data
(60) Provisional application No. 60/632,064, filed on Nov. 30, 2004.

(51) Int. Cl. G01N 33/551

(52) U.S. Cl. 436/524; 977/920

(57) ABSTRACT

Air or gas is drawn into a particle impact collector where the air or gas is forced through non-linear passages inside the collector in such a manner that particles or droplets entrained in the air or gas, or contaminant vapors in the air or gas, are retained in and concentrated by the passages. Once retained and concentrated, the contaminants are suspended or dissolved in a liquid collection medium purging the passages, the collection medium containing assay reagents for a multiplexed binding assay. The agitation produced by the flow of gas and liquid through the non-linear passages increases the reaction rate of the assay.

### CROSS REFERENCE TO RELATED APPLICATION

This application claims benefit from U.S. Provisional Patent Application No. 60/632,064, filed Nov. 30, 2004, the contents of which are incorporated herein by reference in their entirety.

### BACKGROUND OF THE INVENTION

1. Field of the Invention

This invention resides in the field of air sampling and analysis for the presence of contaminants.

2. Description of the Prior Art

Airborne contaminants are of concern in industrial and residential environments, particularly those contaminants that tend to be released in biological warfare. Rather than an analysis for a single agent, simultaneous analyses for multiple agents are needed since it will often be unknown which contaminants are present. Many of the contaminants are biological species such as viruses and microorganisms. The complexities of the analyses for these species typically require extended contact and analysis times and separate units for sample collection and for the assay reactions. This leads to long analysis times, to the extent that a more practical and faster detection method is needed without compromising the ability to detect and analyze a multitude of samples.

### SUMMARY OF THE INVENTION

The present invention resides in the concept of performing multiplexed binding assays on binding members in an aerosol collector, thereby causing the binding reactions involved in the multiplexed analyses to occur in the collector itself. To cause these reactions to occur in the collector, the rinse liquid typically used in the collector is replaced with a liquid suspension or solution of the multiplex assay reagents. The assay reagents are either binding members that are suspended or dissolved in the carrier liquid or themselves bound to solid beads. Multiplexing can thus reside either in the binding members themselves or in beads on which the binding members are immobilized. The surfaces or parts of the aerosol collector that typically retain the airborne or gas-borne particles or other contaminants serve the same function in this invention as they do in the prior art, as well as the additional function of agitating the reaction medium and thereby increasing the degree of contact between the assay reagents and the entrained contaminants drawn from the air or gas by the collector. With this increased contact and agitation, the binding reactions of the assay are completed in a relatively short period of time. Once the binding reactions have occurred, the collection medium is withdrawn from the collector and detection of the analytes is performed in accordance with known and published techniques for multiplexed assays, either in a liquid solution or suspension or on micro-sized or nano-sized beads.

Further objects, features, and advantages of the invention will become apparent from the description that follows.

### DETAILED DESCRIPTION OF THE INVENTION AND PREFERRED EMBODIMENTS

Aerosol collectors, also known as particle impact devices, commonly function by drawing particle-laden atmospheric air or any gaseous stream into a passage that follows a circuitous path causing the entering stream to undergo abrupt changes of direction during its travel. Particulates, droplets, or vapors of relatively high molecular weight in the stream tend to collect on the surfaces of the passage, primarily due to inertial forces, and to thus become concentrated on the surfaces. These concentrated components are then collected by a rinse liquid, which in the present invention contains the multiplex beads and other assay reagents such as the labeled antibodies, and then extracted for analysis. Certain aerosol collectors utilize an external fan to direct the gas stream into the passage. Other aerosol collectors have an impeller incorporated into the body of the collector to draw the air into the collector. In either case, a large volume of air passes through the collector which concentrates the contaminants and disperses them in the liquid to provide a liquid sample with a representative sampling of the contaminants at a high concentration.

Aerosol collectors of this type are described and depicted in Call, P. T., et al. (MesoSystems Technology, Inc.), U.S. Pat. No. 6,267,016 B1, issued Jul. 31, 2001, Moler, C. L., et al. (MesoSystems Technology, Inc.), U.S. Pat. No. 6,729,196 B2, issued May 4, 2004, Saaski, E. W., et al. (Research International, Inc.), U.S. Pat. No. 6,532,835, issued Mar. 18, 2003, and Radolovich, G. (Midwest Research Institute), U.S. Pat. No. 6,925,853, issued Aug. 9, 2005. The contents of these patents are incorporated herein by reference. Examples of commercially available aerosol collectors suitable for use in the practice of this invention are the BIOCAPTURE® 650 Air Sampler and the BIOBADGE™ 100 Air Sampler, both of MesoSystems Technology, Inc., of Kennewick, Wash., USA, the SASS 2000 Plus™ air sampler and ASAP II™ collection/detection system of Research International, Inc., Woodinville, Wash., USA, and the SPINCON® Advanced Air Sampler of Sceptor Industrustries, inc., Kansas City, Mo., USA. The BIOCAPTURE® 650 Air Sampler is a hand-held, battery-operated device that contains a rotating impactor to draw air into the device and to retain particles from the air, a fluid chamber in which the bead and assay reagent suspension can be retained, fluid passages, and a sample collection receptacle that can be analyzed in place or removed and transferred to a separate location or unit for analysis. The BIOCAPTURE® 650 Air Sampler draws air at a rate of over 150 L/min. The BIOBADGE™ 100 Air Sampler is another hand-held, battery-operated device. The BIOBADGE™ 100 Air Sampler is smaller than the BIOCAPTURE® 650 Air Sampler and draws air at a rate of approximately 35 L/min, but is operable in a similar manner with a removable sample collection vial. The rotational speed of rotary aerosol collectors such as the BIOCAPTURE® 650 and BIOBADGE™ 100 Air Samplers affects the size range of airborne particles that are collected, allowing particles in the submicron range to be included. The collection of small particles can be enhanced further by electrostatic forces introduced by applying an electrostatic charge to the collector surfaces. Detection and collection of very small particles can be enhanced even further by introducing a fog to the air being sampled to increase the particle size. The SASS 2000 Plus™ collector is a continuous sampler that transfers particulates in air to a water phase, and the ASAP II™ collection/detection system that incorporates the collector analyzes the captured particulates in a multi-step bioassay that detects up to four analytes. The SPINCON® sampler is a portable device that directs incoming air into a vortex and uses a thin film of stripping liquid to collect particles and vapors from the vortex.

The contaminants collected in the practice of the present invention can be in liquid, solid, or vapor form, and the sample can be air or any gas suspected of containing contaminants. Atmospheric air is of particular interest, and for this reason appears most prominently in the descriptions contained this specification. Liquid contaminants exist as fine droplets suspended in the air. Vapor-phase contaminants are collected by dissolving the vapors in the liquid phase of the bead suspension. Regardless of the phase of the contaminants, the liquid phase of the bead suspension can be water or an aqueous liquid, or an organic liquid such as oils or common organic solvents. The optimal choice of liquid for the liquid phase of the suspension will depend on the types of contaminants. For water-soluble contaminants or contaminants that are readily miscible or dispersible in water, water or aqueous liquids are preferred. A preferred aqueous liquid is buffered saline. Examples of water-soluble contaminants are pinacolyl methylphosphonofluoridate and hydrogen cyanide. Pinacolyl methylphosphonofluoridate, also known as nerve gas, “Soman” and “GD,” is a volatile liquid with a solubility of 2.1% by weight at 20° C., and can be present either as liquid droplets or a vapor. Hydrogen cyanide is a gas that is highly soluble in water. For liquid-phase or vapor-phase contaminants that are not soluble in water or soluble only at very low levels, oils or other organic solvents in which the contaminants are soluble can be used as the liquid phase in the bead suspension.

This invention is useful in general for the collection, concentration, and detection of any substances that are present in the atmosphere, regardless of whether they are produced and/or dispersed by human intervention or by other means, which in suspended form can alter human or animal health or well-being. Examples aside from those listed in the preceding paragraph are infectious agents such as influenza, Bacillus anthracis, Francisella tularensis, Yersinia pestis, Staphylococcus enterotoxin B, botulism toxin A and B, Orthopox virus, Brucella toxin, Bacillus globigii, Erwinia herbicola, ovalbumin, and MS2 virus; small-molecule chemicals such as polyaromatic hydrocarbons, carbon monoxide, mustard gas, and VX; molds such as Stachybotrys and Zygomycetes; toxins such as Botulinium, Ricin, Abrin, and T2; and genetically produced bio-threat agents. Further examples will be readily apparent to those skilled in the art and familiar with the problems of airborne contaminants. Regardless of the level of solubility of the contaminants, the intimate contact between the collection medium and incoming air or the collected contaminants, together with the high degree of agitation that occurs in the collector, will expedite the solubilization of the contaminants in the liquid phase of the suspension and also help dissolve those contaminants with a relatively low solubility. Once dissolved, the contaminants are readily accessible to the surfaces of the multiplex beads for the binding reactions.

When multiplexed binding members are used without a solid phase such as beads, the multiplex feature can be achieved by reporter groups bonded to the binding members, with a different and distinguishable reporter group for each subset. Immunological binding agents are examples of binding members that can be used in this manner, with conventional fluorescent labels, chromogenic labels, or other distinguishable labels serving as the reporter groups

When the multiplexed binding members are binding members immobilized on a solid phase, notably beads of micron or sub-micron size, the multiplex feature can be achieved by characteristics of the beads themselves rather than the binding members bonded to the beads. Aside from the binding reaction between the binding agents and the analytes, the beads are chemically inert to the analytes and any assay materials that the beads will contact during the sampling. The beads are typically polymers, examples of which are polyesters, polyethers, polyolefins, polyalkylene oxides, polyamides, polyurethanes, polysaccharides, celluloses, and polysoprenes. For micron-sized beads, a preferred size range of the bead diameter is from about 1 micron to about 100 microns, and most preferably from about 3 microns to about 30 microns. For nano-sized beads, a preferred size range of the bead diameter is from about 10 nanometers to about 300 nanometers, and most preferably from about 25 nanometers to about 100 nanometers.

The binding members, or beads when used, are divided into subsets and the binding members or beads within any particular subset are homogeneous while those of one subset differ from those of all other subsets in at least one distinguishing characteristic. The number of subsets is not critical and can vary widely. A greater number of subsets will allow the detection of a greater number of analytes. In preferred embodiments of the invention, the number of subsets is in excess of 30, more preferably from about 75 to about 1,000, and most preferably from about 100 to about 500. This enables the subsets to be differentiated by detection instrumentation without separating the subsets from each other. The distinguishing characteristic is preferably at least one of the following: forward light scatter (which generally correlates with bead size and refractive index), side light scatter (which generally correlates with bead size primarily), and fluorescent emission in at least one wavelength, and preferably two or more wavelengths. When beads are used, fluorescence emission as a distinguishing characteristic results from the presence of fluorochromes on the surfaces of the beads or incorporated into the bulk of each bead. With one or more of these characteristics serving as distinguishing characteristics, the subset to which any bead belongs can be identified by flow cytometry according to known techniques. In particularly preferred embodiments, the distinguishing characteristic is fluorescence emission, and combinations of two or more fluorochromes with emission maxima at different wavelengths can be used to distinguish large numbers of subsets by variations in the proportions of the fluorochromes. With two fluorochromes, for example, in which each is incorporated in the beads at one of ten different concentrations, 100 distinctive combinations can be formed, thereby allowing the use of 100 distinguishable bead subsets. Other methods of selecting and manipulating the characteristics to achieve combinations and large numbers of subsets will be readily apparent to those skilled in the art. Microbeads with dyes incorporated are commercially available from suppliers, including Spherotech, Inc. (Libertyville, Ill., USA), Molecular Probes, Inc. (Eugene, Oreg., USA), and Luminex Corporation (Austin, Tex., USA). The use of multiplexing beads as described in this paragraph is further described in Chandler, V. S., et al. (Luminex Corporation), U.S. Pat. No. 6,411,904 B1 (issued Jun. 25, 2002), and Chandler, V. S., et al. (Luminex Corporation), U.S. Pat. No. 6,449,562 B1 (issued Sep. 10, 2002). The contents of each of these patents is incorporated herein by reference.

For use in the collector, the binding members or beads (when present) of the various subsets are pooled into a single solution or suspension in a common carrier liquid. When beads are used, each subset, prior to the pooling of the subsets, is individually coupled to a binding agent that is specific for a single contaminant among those to be detected. Immunologically specific antibodies that have been developed for particular analytes (i.e., contaminants) can be coupled to the bead surface by conventional coupling techniques. These coupling techniques may involve electrostatic attraction, specific affinity interaction, hydrophobic interaction, or covalent binding. Covalent binding is preferred, using either functional groups on the bead surface or linking groups between the bead and the binding agent. Examples of suitable functional groups are amine groups, ammonium groups, hydroxyl groups, carboxylic acid groups, and isocyanate groups, any of which can be introduced into polymeric beads by the use of functionalized monomers in the polymerization processes to form the beads. When linking groups are used, they may provide multiple binding sites to increase the density of binding sites on an individual bead as well as to reduce steric hindrance among multiple analyte molecules binding to a single bead, in either case increasing the range and sensitivity of the assay. Linking groups can also add specific types of reactive groups to the beads that are not otherwise incorporated into the bead structure. Examples of multi-binding-site linking groups are polylysine, polyaspartic acid, polyglutamic acid and polyarginine. Carboxylated microbeads to which these linking groups are readily bonded are available from the suppliers listed above.

The binding agents themselves will vary with the analytes being detected and the type of assay to be performed. One preferred class of binding agents, as noted above, is immunological binding agents. These include antibodies, antigens, haptens, and other specific binding proteins such as biotin and avidin. Among the various types of assays are competitive assays and immunometric or enzyme-linked immunosorbent assays (ELISAs), including sandwich assays. The protocols of these assays are well known among skilled immunologists. In sandwich assays and ELISAs, the binding agent immobilized on the beads is an antibody to the analyte, and the bound antibodies are present in excess relative to the suspected quantity range of the analyte so that all analyte in the sample binds. A second antibody to the analyte is also present in the suspension as a second assay reagent, the second antibody labeled with a detectable label, preferably a fluorescent label that will allow detection of the presence of the analyte and quantification. When fluorescent emission is used both as the distinguishing characteristic of the bead subset and the label on the second antibody, the fluorochromes can be selected so that their emission maxima are sufficiently far apart from each other that they can be detected independently. Other preferred classes of binding agents and binding chemistry are oligonucleotides and oligonucleotide binding chemistry, and basic protein binding chemistry. The chemical structures of the binding members in each and the bonds themselves are well known among those skilled in the technologies of oligonucleotide chemistry and protein chemistry.

Labels are thus used as a means of detecting the amount of bound analyte resulting from the binding reaction or as a means of differentiating among the different subsets, or both. Labels used for either purpose can be any label that is capable of emitting a detectable signal. Fluorophores and colorimetric labels are preferred. When beads are used, these labels can also be incorporated into the beads themselves as distinguishing characteristics that differentiate one subset from the next. Fluorophores and chromogenic labels suitable for either use are widely reported in the literature and thus known to those skilled in the art, and many are readily available from commercial suppliers to the biotechnology industry. Literature sources for fluorophores and chromogenic labels include Cardullo et al., Proc. Natl. Acad. Sci. USA 85: 8790-8794 (1988); Dexter, D. L., J. of Chemical Physics 21: 836-850 (1953); Hochstrasser et al., Biophysical Chemistry 45: 133-141 (1992); Selvin, P., Methods in Enzymology 246: 300-334 (1995); Steinberg, I., Ann. Rev. Biochem., 40: 83-114 (1971); Stryer, L., Ann. Rev. Biochem. 47: 819-846 (1978); Wang et al., Tetrahedron Letters 31: 6493-6496 (1990); Wang et al., Anal. Chem. 67: 1197-1203 (1995).

The following are examples of these labels:
4-acetamido-4′-isothiocyanatostilbene-2,2′disulfonic acid
acridine
acridine isothiocyanate
5-(2′-aminoethyl)aminonaphthalene-1-sulfonic acid (EDANS)
4-amino-N-[3-vinylsulfonyl)phenyl]naphthalimide-3,5 disulfonate
N-(4-anilino-1-naphthyl)maleimide
anthranilamide
BODIPY
Brilliant Yellow
coumarin
7-amino-4-methylcoumarin (AMC, Coumarin 120)
7-amino-4-trifluoromethylcoumarin (Coumaran 151)
cyanine dyes
cyanosine
4′,6-diaminidino-2-phenylindole (DAPI)
5′,5″-dibromopyrogallol-sulfonaphthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansylchloride)
4-(4′-dimethylaminophenylazo)benzoic acid (DABCYL)
4-dimethylaminophenylazophenyl-4′-isothiocyanate (DABITC)
eosin
eosin isothiocyanate
erythrosin B
erythrosin isothiocyanate
ethidium
5-carboxyfluorescein (FAM)
5-(4,6-dichlorotriazin-2-yl)aminofluorescein (DTAF)
2′,7′-dimethoxy-4′5′-dichloro-6-carboxyfluorescein (JOE)
fluorescein
fluorescein isothiocyanate
fluorescamine
gold sol
IR144
IR1446
Malachite Green isothiocyanate
4-methylumbelliferone
ortho cresolphthalein
nitrotyrosine
pararosaniline
Phenol Red
B-phycoerythrin
o-phthaldialdehyde
platinum sol
pyrene
pyrene butyrate
succinimidyl 1-pyrene butyrate
quantum dots
Reactive Red 4 (Cibacron™ Brilliant Red 3B-A)
6-carboxy-X-rhodamine (ROX)
6-carboxyrhodamine (R6G)
lissamine rhodamine B sulfonyl chloride rhodamine (Rhod)
rhodamine B
rhodamine 123
rhodamine X isothiocyanate
selenium sol
silver sol
sulforhodamine B
sulforhodamine 101
sulfonyl chloride derivative of sulforhodamine 101 (Texas Red)
tellurium sol
N,N,N′,N′-tetramethyl-6-carboxyrhodamine (TAMRA)
tetramethyl rhodamine
tetramethyl rhodamine isothiocyanate (TRITC)
riboflavin
rosolic acid
lanthanide chelate derivatives

Different labels can be used in combination, with a distinct label for each analyte. Preferably, however, a single label is used for all labeled binding members, the assays being differentiated solely by the distinguishing characteristic that distinguishes the individual bead subsets from each other. Methods of attachment of a label to the binding member are well known in the art.

When beads are used, multiplex analysis of the beads subsequent to the reaction can be performed by any methods known to be effective for pooled subsets of beads. One method is flow cytometry. Flow cytometry involves the passage of a suspension of the beads as a stream past a light beam and electro-optical sensors in such a manner that only one bead at a time passes the sensors. Each bead passing this region perturbs the light beam, and the resulting scattered or emitted light are detected. The subset for each bead is then identified by the distinguishing characteristic, which is generally an optical signal, along with a separate optical signal for the presence and amount of label, thereby producing individual assay results. Descriptions of instrumentation and methods for flow cytometry are found in the literature. Examples of literature references on the subject are McHugh, “Flow Microsphere Immunoassay for the Quantitative and Simultaneous Detection of Multiple Soluble Analytes,” Methods in Cell Biology 42, Part B (Academic Press, 1994); McHugh et al., “Microsphere-Based Fluorescence Immunoassays Using Flow Cytometry Instrumentation,” Clinical Flow Cytometry, Bauer, K. D., et al., eds. (Baltimore, Md., USA: Williams and Williams, 1993), pp. 535-544; Lindmo et al., “Immunometric Assay Using Mixtures of Two Particle Types of Different Affinity,” J. Immunol. Meth. 126: 183-189 (1990); McHugh, “Flow Cytometry and the Application of Microsphere-Based Fluorescence Immunoassays,” Immunochemica 5: 116 (1991); Horan et al., “Fluid Phase Particle Fluorescence Analysis: Rheumatoid Factor Specificity Evaluated by Laser Flow Cytophotometry,” Immunoassays in the Clinical Laboratory, 185-189 (Liss 1979); Wilson et al., “A New Microsphere-Based Immunofluorescence Assay Using Flow Cytometry,” J. Immunol. Meth. 107: 225-230 (1988); Fulwyler et al., “Flow Microsphere Immunoassay for the Quantitative and Simultaneous Detection of Multiple Soluble Analytes,” Meth. Cell Biol. 33: 613-629 (1990); Coulter Electronics Inc., United Kingdom Patent No. 1,561,042 (published Feb. 13, 1980); and Steinkamp et al., Review of Scientific Instruments 44(9): 1301-1310 (1973).

An alternative method for multiplex analysis of the binding members or beads is by the use of strips on which specific binding agents are affixed. Such strips are available from Tetracore, Inc., Gaithersburg, Md., USA.

What is claimed is:

1. A process for the detection and quantification of a plurality of analytes in a gas, said process comprising:
(a) drawing a stream of said gas into a collector and causing said stream thus drawn to pass through a non-linear passage inside said collector while analytes are retained in said passage,
(b) purging said passage with a collection medium comprising a carrier liquid and multiplexed binding members comprised of a plurality of subsets of said binding members, each subset distinguishable from all other subsets by a characteristic that is distinguishable by instrumentation and each comprising a distinctive binding member that selectively binds one analyte of said plurality of analytes, and
(c) detecting the binding of analytes to said binding members in a manner that differentiates among said subsets.

2. The process of claim 1 wherein said multiplexed binding members are beads to which said binding members are bound, and said distinguishable characteristic is a characteristic of said beads.

3. The process of claim 1 wherein said multiplexed binding members are immunological binding members that are not immobilized on a solid phase, and said distinguishable characteristic are reporter molecules bound to said immunological binding members, a distinctive reporter molecule for each said subset.

4. The process of claim 1 wherein said analytes are water-soluble and said carrier liquid is an aqueous liquid.

5. The process of claim 1 wherein said analytes are soluble in an organic liquid and said carrier liquid is an organic liquid.

6. The process of claim 1 wherein said distinguishable characteristic is a colorimetric label.

7. The process of claim 1 wherein said distinguishable characteristic is a fluorescent label.

8. The process of claim 1 wherein step (c) produces analyte-binding member complexes, and step (d) comprises binding a colorimetric label to said analyte-binding member complexes and detecting the amount of said label so bound.

9. The process of claim 1 wherein step (c) produces analyte-binding member complexes, and step (d) comprises binding a fluorescent label to said analyte-binding member complexes and detecting the amount of said label so bound.

10. The process of claim 1 wherein said gas is air.

11. The process of claim 2 wherein said beads are from about 1 micron to about 100 microns in diameter.

12. The process of claim 2 wherein said beads are from about 3 microns to about 30 microns in diameter.

13. The process of claim 2 wherein said beads are from about 10 nanometers to about 300 microns in diameter.

14. The process of claim 2 wherein said beads are from about 25 nanometers to about 100 microns in diameter.

15. The process of claim 2 wherein said multiplexed binding members consist of greater than 30 said subsets.

16. The process of claim 2 wherein said multiplexed binding members consist of from about 75 to about 1,000 said subsets.

17. The process of claim 2 wherein said multiplexed binding members consist of from about 100 to about 500 said subsets.

18. The process of claim 2 wherein said multiplexed beads consist of greater than 30 said subsets.

19. The process of claim 2 wherein said multiplexed beads consist of from about 75 to about 1,000 said subsets.

20. The process of claim 2 wherein said multiplexed beads consist of from about 100 to about 500 said subsets.
